(19) United States
(12) Patent Application Publication
(10) Pub. No.: US 2008/0295842 A1
(43) Pub. Date: Dec. 4, 2008

(54) Method and Apparatus for Simplified Precise Mechanical Gas Mixing and Delivery for Animal Research

(75) Inventor: Bernard F. Hete, Kittanning, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR LIFE SCIENCES CORP., Oakmont, PA (US)

(21) Appl. No.: 12/129,643

(22) Filed: May 29, 2008

Related U.S. Application Data
(60) Provisional application No. 60/940,552, filed on May 29, 2007.

(51) Int. Cl. A61M 16/10; A61M 16/20; A62B 7/02; A62B 9/02

(52) U.S. Cl. 128/205.24; 128/204.18; 128/205.11

(57) ABSTRACT

An apparatus for supplying a precise oxygen-nitrogen gas mixture to an animal comprises (i) A source of pressurized oxygen gas, (ii) A source of pressurized nitrogen gas, (iii) A mechanical two-gas blending valve having a first and second gas inlet couplings, and an outlet coupling, the gas blending valve having a manual selector that can incrementally adjust the oxygen-nitrogen gas mixture within a range of mixtures, and a face plate on the blending valve including markings adjacent to the manual selector that are indicative of specific oxygen-nitrogen mixtures, wherein the gas inlets are coupled to the sources of oxygen and nitrogen, respectively, (iv) A gas dispenser coupled to the outlet of the mechanical two gas blending valve; and (v) An animal interface coupling to deliver the gas mixture to the subject animal. The apparatus may be provided for mixtures of air and any gas of interest.

### RELATED APPLICATIONS

This application claims the benefit of Provisional Patent Applications Ser. No. 60/940,552 filed May 29, 2007 entitled “Method and Apparatus for Simplified Precise Mechanical Gas Mixing and Delivery for Animal Research.”

### BACKGROUND OF THE INVENTION

1. Field of the Invention

The present invention relates to a method and apparatus for precision gas mixing and delivery for animal research, more particularly, the present invention provides a method and apparatus for precise oxygen/nitrogen and other gas mixing and delivery for animal studies.

2. Background Information

In animal studies, such as hypoxia studies, the mixing of respiratory gases to be delivered to the subjects is an arduous setup for the study. One common approach is to develop a precise mixing valve arrangement with metered flow valves leading to a blending “T”, whereby the researchers calculate the desired flow rates for the specific constituents to obtain the desired gas mixture. In addition to the time consuming issue, calculation errors can lead to an invalidation of the study results.

Another common approach is to limit the studies to pre-packaged mixtures that are supplied by others (e.g. Compressed Air, 18% O2/82% N, 15% O2/85% N, etc). This approach simplifies the gas mixing step but limits the data points that are available and is simply unacceptable for many studies.

Researchers P. A. Robbins, G. D. Swanson, A. J. Micco and W. P. Schubert developed a “fast gas-mixing system for breath-to-breath respiratory control studies” described in Journal of Applied Physiology, Vol 52, Issue 5 1358-1362, 1982. This is a computer-controlled gas-mixing system that manipulates inspired CO2 and O2 on a breath-to-breath basis, wherein the system uses pairs of solenoid valves, one pair for each gas. The valves cycle open and shut every 1/12 s. A circuit converts signals from the computer, which dictates the flows of the gases, into a special form for driving the valve pairs. These signals determine the percentage of time within the 1/12-s cycle each valve spends in an open state and the percentage of time it spends shut, which, in effect, sets the average flows of the various gases to the mixing chamber. The delay for response of the system to commanded CO2 or O2 changes is less than 200 ms.

CWE Inc. has developed the GSM-3 Gas Mixer for creating custom respiratory gas mixtures. Any three gases can be connected as inputs, and the output is the user-programmed mixture of these gases. The instrument can operate in a stand-alone mode, with mixture programming done from the front panel controls, or it can be controlled using an attached computer running the supplied software. The mixer uses thermal mass flow controllers to provide any programmed mixture in the range of 0-10 lpm for each gas with a resolution of 0.1% concentration. Front panel controls are used to set the desired total output flow, as well as the concentration of each of the three component gases. The LCD display shows the set concentrations, as well as the computed flow rates for each gas. Up to four custom mixtures can be created and stored in non-volatile memory for future use. Any of these stored mixtures can be immediately selected and executed. This function allows rapid step-changes in concentrations to be performed with the push of a button. The flow controllers are pre-calibrated for the three most commonly used respiratory gases: O2, CO2, and N2. Other gases can also be selected, and internal correction factors are automatically applied to compensate the flow controllers for differing gas densities. The GSM-3 Gas Mixer comes ready to use with CDROM software and a serial cable.

These computerized, digital gas mixing devices still represent several problems for the researchers. The first is that these systems can be priced beyond the researcher's budget for particular studies and may not be useful in that they are (due to costs constraints) not made readily available to the researchers. Secondly they add too much complexity to the gas mixing procedure, although they do provide for accuracy (assuming the researchers do not hook the systems up incorrectly).

There remains a need in the art for a simple-to-operate, intuitive, accurate, precise gas mixing method and apparatus for animal studies.

### SUMMARY OF THE INVENTION

One embodiment of the present invention provides an efficient apparatus for supplying a precise oxygen-nitrogen gas mixture to an animal comprises (i) A source of pressurized oxygen gas, (ii) A source of pressurized nitrogen gas, (iii) Regulators on each gas source to regulate the pressure of each gas, (iv) A mechanical two gas blending valve having a first gas inlet coupling, a second distinct gas inlet coupling and an outlet coupling configured for dispensing an oxygen-nitrogen mixture, the gas blending valve having a manual selector that can incrementally adjust the oxygen-nitrogen gas mixture within a range of mixtures, and a face plate on the blending valve including markings adjacent to the manual selector that are indicative of specific oxygen-nitrogen mixtures, or given percentages of either or both gases, wherein the gas inlets are coupled to the sources of oxygen and nitrogen, respectively, (v) A gas dispenser coupled to the outlet of the mechanical two-gas blending valve; and (vi) An animal housing configured to contain an animal therein, wherein the gas dispenser is coupled to the animal housing and configured to dispense the oxygen-nitrogen gas mixture to the animal housing, or any interface coupling to deliver the gas mixture to the airway of the subject directly, or through another device such as a mechanical ventilator

The present invention further includes a method comprising the additional steps of coupling the source of oxygen gas to a gas inlet of the blending valve; coupling the source of nitrogen to a gas inlet of the blending valve; Manually adjusting the selector to select a given oxygen-nitrogen gas mixture; and simultaneously supplying oxygen gas to the blending valve, whereby the blending valve will dispense the oxygen-nitrogen mixture to the dispenser that will dispense the oxygen-nitrogen mixture to the animal within the animal housing, or to any other airway interface.

In some non-limiting embodiments of the present invention the first gas inlet coupling is coupled only to the source of oxygen and the second gas inlet coupling is coupled only to the source of nitrogen. One non-limiting aspect of this embodiment provides that the source of oxygen is a compressed oxygen container and the source of nitrogen is a compressed nitrogen container wherein the face plate includes markings adjacent the manual selector that are indicative of specific oxygen-nitrogen mixtures from 0% oxygen to 100% oxygen. Another non-limiting aspect of this embodiment provides that the source of oxygen is a compressed air container and the source of nitrogen is a compressed nitrogen container and wherein the face plate includes markings adjacent the manual selector that are indicative of specific oxygen-nitrogen mixtures from 0% oxygen to 21% oxygen.

In some non-limiting embodiments of the present invention further include three containers including a compressed air container, a compressed nitrogen container and a compressed oxygen container, wherein two of the three containers are selectively coupled to the blending valve to provide the source of oxygen and the source of nitrogen to the blending valve, whereby an oxygen-nitrogen selection provides for a gas mixture range of 0% Oxygen to 100% Oxygen on the manual selector, an air-nitrogen selection provides for a gas mixture range of 21% oxygen to 0% Oxygen, and an Air-Oxygen selection provides for a gas mixture range of 21% Oxygen to 100% Oxygen.

One nonlimiting aspect of the invention provides a method of supplying an air and subject gas of interest gas mixtures between 0% gas of interest and X% gas of interest to an animal, wherein X is less than 100%. This method comprises the steps of: (i) Providing a source of pressurized air; (ii) Providing a source of pressurized gas of interest, wherein the source of gas of interest is a preformed mixture of X% gas of interest and the remainder air; (iii) Providing a mechanical two gas blending valve having a first gas inlet coupling, a second distinct gas inlet coupling and an outlet coupling configured for dispensing an air-gas of interest mixture, the gas blending valve having a manual selector that can incrementally adjust the gas mixture within a range of mixtures, and a face plate on the blending valve including markings adjacent to the manual selector that are indicative of specific air-gas of interest mixtures between 0% and X% gas of interest concentrations; (iv) Providing a gas dispenser coupled to the outlet of the mechanical two gas blending valve; (v) Providing an animal interface coupled to the gas dispenser and configured to deliver the gas mixture to the subject animal; (vi) Coupling the source of air to a gas inlet of the blending valve; (vii) Coupling the source of gas of interest to a gas inlet of the blending valve; (viii) Manually adjusting the selector to select a given gas mixture; (ix) Simultaneously supplying air and gas of interest containing gas to the blending valve, whereby the blending valve will dispense the selected gas mixture to the dispenser that will dispense the gas mixture to the animal through the animal interface.

These and other advantages of the present invention will be clarified in the brief description of the preferred embodiment taken together with the drawings in which like reference numerals represent like elements throughout.

### BRIEF DESCRIPTION OF THE DRAWINGS

FIG. 1 is a schematic view of an oxygen-nitrogen gas mixing apparatus for animal research according to one aspect of the present invention;

FIG. 2 is a schematic view of an air-nitrogen gas mixing apparatus for animal research according to one aspect of the present invention;

FIG. 3 is a schematic view of an oxygen-nitrogen-air gas mixing apparatus for animal research according to one aspect of the present invention; and

FIG. 4 is a schematic view of a multiple gas mixing apparatus for animal research according to one aspect of the present invention;

FIGS. 5A and 5B are schematic views of particular face plates for gas mixing apparatuses for animal research according to aspects of the present invention; and

FIG. 6 is a schematic view of an air/gas-of-interest gas mixing apparatus for animal research according to one aspect of the present invention.

### DESCRIPTION OF THE PREFERRED EMBODIMENTS

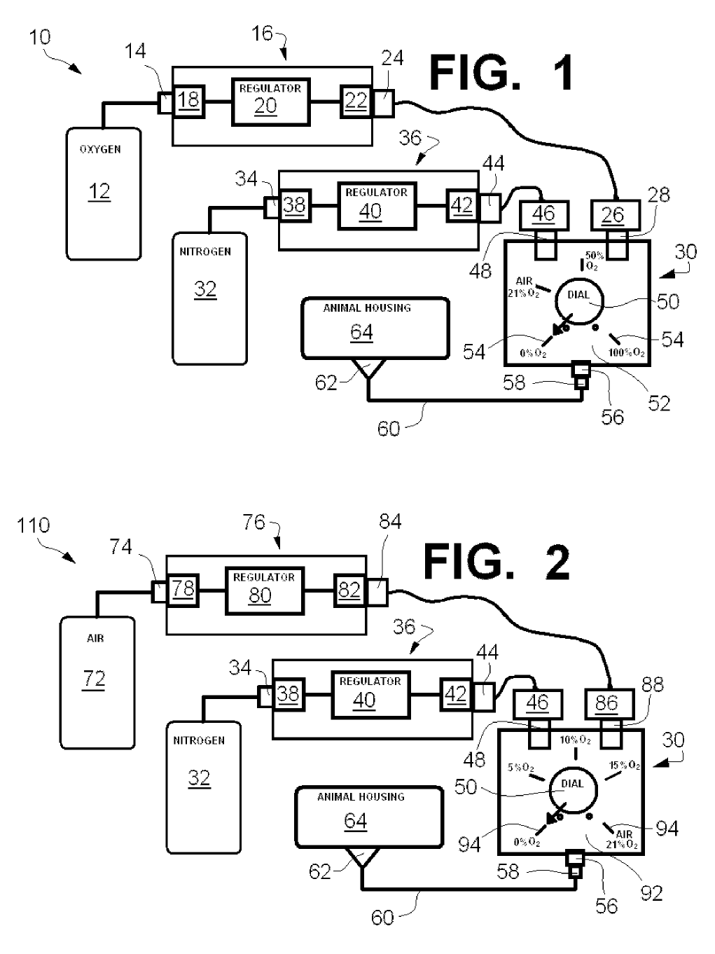

FIG. 1 is a schematic view of an oxygen-nitrogen gas mixing apparatus 10 for animal research according to one aspect of the present invention that will provide specific oxygen-nitrogen mixtures from 0% oxygen to 100% oxygen.

The apparatus 10 includes a source of pressurized oxygen gas in the form of a compressed oxygen gas container 12, or canister, that is widely commercially available from gas suppliers. The container 12 should meet the specifications and color markings of an Oxygen tank, as established by the Compressed Gas Association. The container 12 includes an oxygen tank fitting 14, also called a CGA—oxygen gas fitting. CGA stands for “Compressed Gas Association” which is the group that established standards in the gas industry for fittings which are used to attach to specific gas cylinders. For nearly a century, the Compressed Gas Association has been dedicated to the development and promotion of safety standards and safe practices in the industrial gas industry. CGA fitting standards are developed through the combined efforts of more than 200 member companies worldwide. In general, these CGA fitting connections are designed for metal-to-metal sealing, however a sealing washer may be provided on those fittings that do not have a metal-to-metal set.

The fitting 14 couples to a regulator assembly 16 associated with the oxygen container 12. The regulator assembly 16 is equipped with a CGA fitting 18 that matches its intended gas service, namely oxygen gas, and thus matches fitting 14. The regulator assembly 16 includes a conventional gas regulator 20 for the oxygen gas to regulate the pressure of the outgoing gas. The outlet of the regulator assembly 16 is a CGA fitting 22 that is specific to oxygen.

A coupling hose with a CGA fitting 24 specific to oxygen is attached to the CGA fitting 22 of the regulator assembly and opposite CGA fitting 26 specific to oxygen is coupled to a CGA fitting 28 specific to oxygen found on a two-gas mechanical blending valve 30.

Two gas mechanical blending valves, per se, are known in the medical respiratory fields such as the M2100 Air/O2 Blender from General Electric, Inc. that is designed to blend compressed air and oxygen, and to deliver pressurized gas at precise concentrations. The quality and reliability of Bird® blenders from Viasys Healthcare, Inc. make these two gas mechanical blenders also acceptable for forming the general working structure for the valve 30. The main modifications needed to these off-the-shelf components is the provision of specific CGA fittings that prevent the researcher from “hooking the system up backwards”, and the calibration of the blender for the oxygen nitrogen mixture from 0% Oxygen to 100% Oxygen as found in the embodiment of FIG. 1.

The apparatus 10 includes a source of pressurized nitrogen gas in the form of a compressed nitrogen gas container 32, or canister, that is also widely commercially available from gas suppliers. The container 32 should meet the specifications and color markings of a Nitrogen tank, as established by the Compressed Gas Association. The container 32 includes a nitrogen tank fitting 34, also called a CGA—nitrogen gas fitting.

The fitting 34 couples to a regulator assembly 36 associated with the nitrogen container 32. The regulator assembly 36 is equipped with a CGA fitting 38 that matches its intended gas service, i.e. nitrogen, and thus matches fitting 34. The regulator assembly 36 includes a conventional gas regulator 40 for the nitrogen gas to regulate the pressure of the outgoing gas. The outlet of the regulator assembly 36 is a CGA fitting 42 that is specific to nitrogen.

A coupling hose with a CGA fitting 44 specific to nitrogen is attached to the CGA fitting 42 of the regulator assembly 36 and opposite CGA fitting 46 specific to nitrogen is coupled to a CGA fitting 48 specific to nitrogen found on a two-gas mechanical blending valve 30.

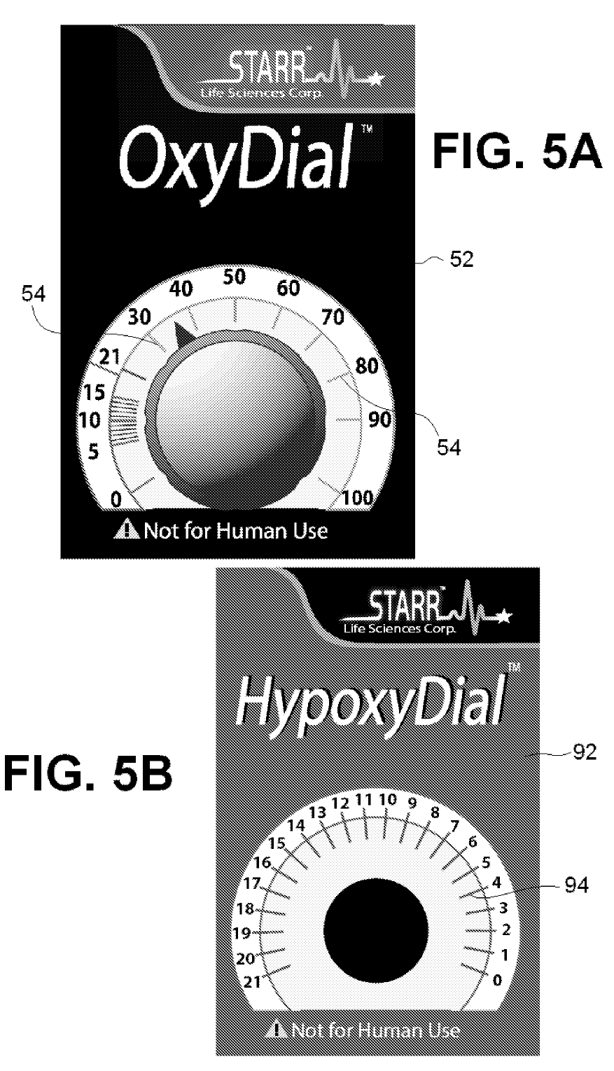

The mechanical two-gas blending valve 30 includes a manually adjustable selector 50 such as a rotary dial with an indicator arm identifying the position. Stops, such as pins, can limit the rotation of the manual selector at the particular extremes. The extreme positions will effectively be at an oxygen-nitrogen mixture of 0% oxygen at one extreme and 100% oxygen at the other extreme, and the intermediate positions will allow the researcher to easily, and precisely select any desired mixture. The face plate 52 of the valve 30 has markings or indicia 54 at specific locations to give the researcher the necessary reference. Only a few indicia points or markings 54 are shown, but as many as desired can be provided to assist the researcher. One particularly useful marking is at 21% oxygen, which is largely the equivalent of Air. One could also mark the faceplate with numbers corresponding to the nitrogen content of the gas rather than the oxygen content, but this would be less useful to an animal researcher given that oxygen is the primary gas of interest. FIG. 5A illustrates an appropriate face plate 52 construction including dial 50.

The outlet of the valve 30 is a CGA fitting 56. The fitting 56 is preferably different from inlet fittings 28 and 48 to avoid any miss-assembly by the researcher. A CGA fitting 58 matching fitting 56 is on coupling hose 60 that extends to a gas dispenser 62 that is attached to an animal housing 64 configured to contain an animal therein, wherein the gas dispenser 62 is configured to dispense the oxygen-nitrogen gas mixture to the animal housing 64. The gas dispenser 62 and animal housing 64 can take a number of forms as known in the art.

The apparatus 10 allows a researcher to quickly, easily, repeatably and cost-effectively provide any desired Oxygen-Nitrogen mixtures to animals for a variety of studies. The use of specific CGA fittings prevents the researcher from incorrectly assembling the system. The use of the blender valve 30 is intuitive and the markings 54 will give the researcher the precise control needed. Further the CGA components make the system safe for use in research environments.

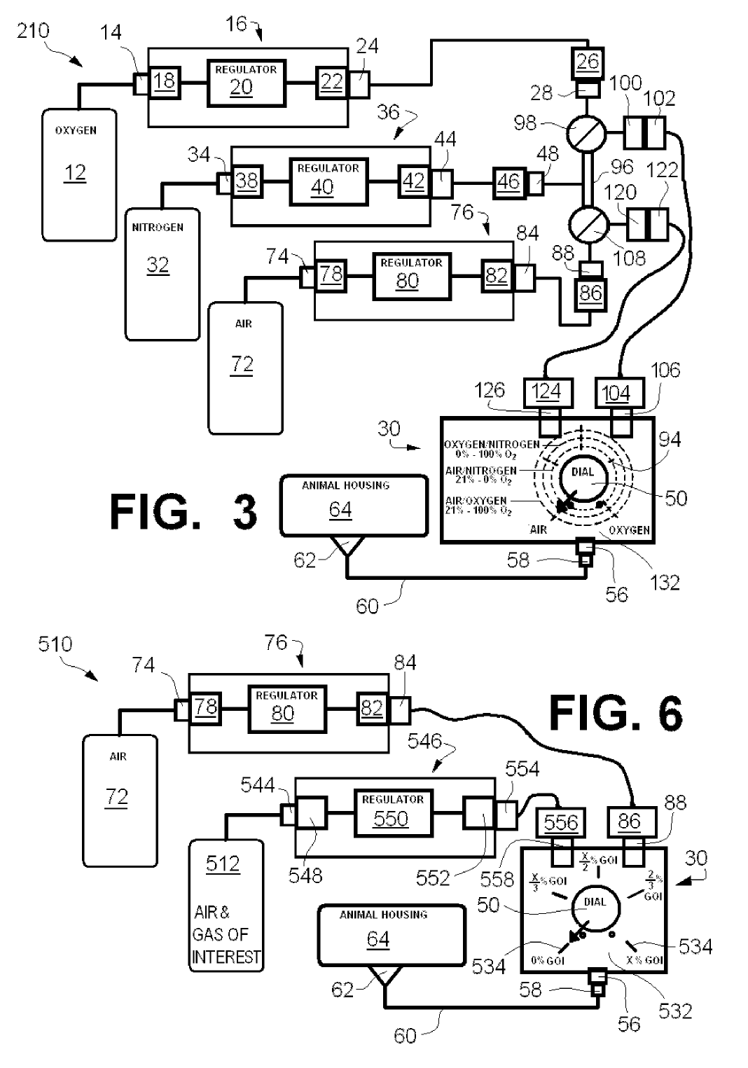

The apparatus 10 allows a researcher to perform hypoxia studies on animals, however, in such studies the range of control for the valve 30 is from 21% oxygen to 0% Oxygen with the selector 50. It is worthwhile to note that hypoxia is an inadequacy in the oxygen reaching the body's tissues. For these specific studies, the apparatus 110 of FIG. 2 may be more advantageous.

The apparatus 110 is similar to apparatus 10 described above wherein like reference numerals represent like elements, and these repeated elements need not be described again. The main difference is that the source of oxygen in this system is from a compressed air container or canister 72, replacing container 12 of apparatus 10.

The container 72 should meet the specifications and color markings of a compressed air tank, as established by the Compressed Gas Association. The container 72 includes an air tank fitting 74, also called a CGA—air gas fitting. The fitting 74 couples to a regulator assembly 76 associated with the air container 72. The regulator assembly 76 is equipped with a CGA fitting 78 that matches its intended gas service, i.e. air, and thus matches fitting 74. The regulator assembly 76 includes a conventional gas regulator 80 for the air gas to regulate the pressure of the outgoing gas. The outlet of the regulator assembly 76 is a CGA fitting 82 that is specific to air.

A coupling hose with a CGA fitting 84 specific to air is attached to the CGA fitting 82 of the regulator assembly 76 and opposite CGA fitting 86 specific to air is coupled to a CGA fitting 88 specific to air found on the two-gas mechanical blending valve 30.

Another important difference with apparatus 110 is that the face place 52 is replaced with another appropriate face plate 92. The face plate 92 of the valve 30 has markings or indicia 94 at specific locations to give the researcher the necessary reference. Only a few indicia points or markings 94 are shown, but as many as desired can be provided to assist the researcher. The extreme positions will effectively be at an oxygen-nitrogen mixture of 0% oxygen at one extreme and 21% oxygen (i.e. Air) at the other extreme, and the intermediate positions will allow the researcher to easily, and precisely select any desired mixture. FIG. 5B illustrates a face plate 92 for the apparatus 110. The apparatus 110 allows the researcher a greater control of specific settings for ranges of 0-21% Oxygen.

A further embodiment of the present invention is shown in FIG. 3 with apparatus 210. The apparatus 210 combines the advantages of the apparatus 10 and 110 and adds more functionality, at the cost of making the system slightly more complex. The apparatus 210 is similar to apparatus 10 and 110 described above wherein like reference numerals represent like elements, and these repeated elements need not be described again. The main difference is that the apparatus 210 provides for three containers, including the compressed air container 72, the compressed nitrogen container 32 and the compressed oxygen container 12, wherein two of the three containers 12, 32 and 72 are selectively coupled to the blending valve 30 to provide the source of oxygen and the source of nitrogen to the blending valve 30. In this apparatus 210 an oxygen-nitrogen selection provides for a gas mixture range of 0% Oxygen to 100% Oxygen on the manual selector 50, an air-nitrogen selection provides for a gas mixture range of 21% oxygen to 0% Oxygen, and an Air-Oxygen selection provides for a gas mixture range of 21% Oxygen to 100% Oxygen.

Specifically, the oxygen CGA fitting 28 leads to the inlet of a selector valve 98, while the nitrogen CGA fitting 48 leads to a T-coupling that is attached to the selector valve 98, whereby the selector valve can select oxygen or nitrogen to move forward through CGA fitting 100. The preference for fitting 100 is that it be different from the remaining CGA fittings such that the apparatus only has one way that it can be assembled by the researcher. A coupling hose with a CGA fitting 102 is attached to the matching CGA fitting 100 of the selector valve 98 and opposite CGA fitting 104 is coupled to a matching CGA fitting 106 found on the two-gas mechanical blending valve 30. Again, preferably the fittings 104 and 106 are selected such that the apparatus is assembled in one correct orientation.

The air CGA fitting 88 leads to the inlet of a selector valve 108, while the nitrogen CGA fitting 48 leads to a T-coupling that is attached to the selector valve 98 as noted above and to the selector valve 108 as shown, whereby the selector valve 108 can select air or nitrogen to move forward through CGA fitting 120. The preference for fitting 120 is that it be different from the remaining CGA fittings such that the apparatus 210 only has one way that it can be assembled by the researcher. A coupling hose with a CGA fitting 122 is attached to the matching CGA fitting 120 of the selector valve 108 and opposite CGA fitting 124 is coupled to a matching CGA fitting 126 found on the two gas mechanical blending valve 30. Again, preferably the fittings 124 and 126 are selected such that the apparatus is assembled in one correct orientation.

It should be apparent that the selector valves 98 and 108 allow for the selection of the Oxygen container 12 and the Nitrogen container 32, or the Oxygen container 12 and the Air container 72, or the Air container 72 and the Nitrogen container 32. Technically the valves, as shown, will also allow for a Nitrogen-Nitrogen mixture to be selected, but that is hardly a meaningful combination. Further the face plate 132 includes markings such as 54 and 94, on different radial bands that are associated with each meaningful combination of gases that can be selected. The apparatus 210 provides obvious additional flexibility to the researcher, but it adds the requirement that the researcher be well aware of the specific selections of the selector valves 98 and 108. The additional flexibility and increased complexity is reflected in the markings on the faceplate. One possible modification is including sensors on the selector valves 98 and 108 that could be monitored and displayed on the face plate by lighting up the appropriate markings on the face plate 132, however this type of modification is technically feasible, but difficult to accomplish in a cost-effective manner, and begins to move away from the goals of the present invention.

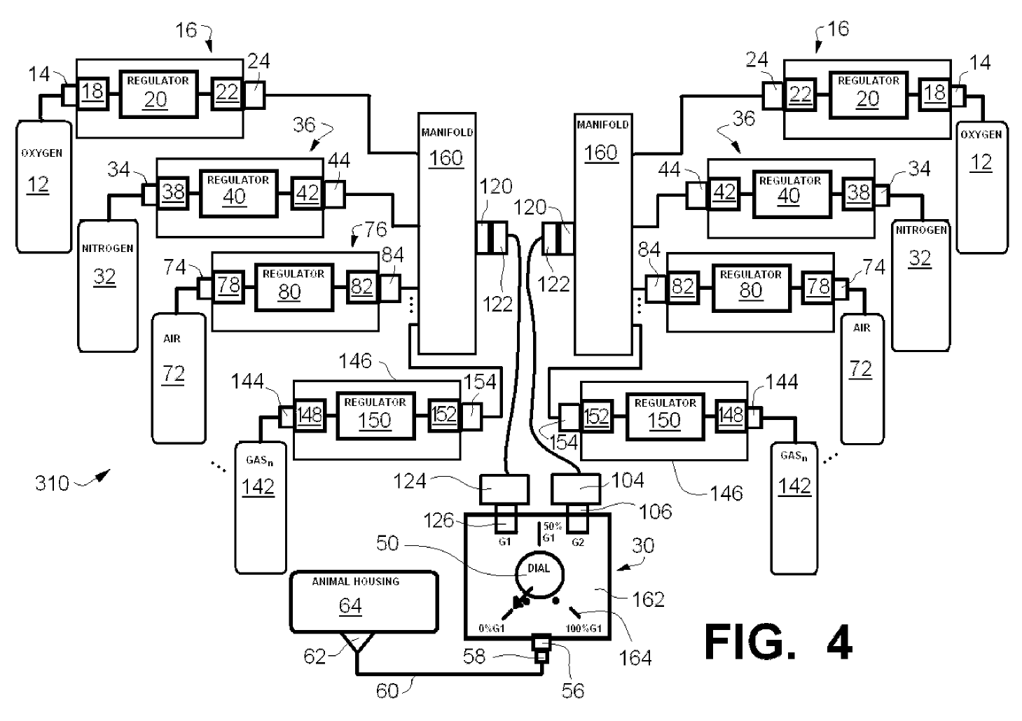

Another embodiment of the present invention is to expand upon the flexibility offered with the system of FIG. 3. FIG. 4 illustrates a system that can accommodate any number of gases. The gases could be pre-packaged mixtures of oxygen and nitrogen in addition to air or can be other gases, or mixtures thereof. In this embodiment the selector valves (or three-way valves) 98 and 108 are replaced with two manifolds 160 each having the specific inputs therein, including the Oxygen container 12, the nitrogen container 32 the air container 72. Additional gases are reflected with gas container 142 (representing gas n) with CGA coupling 144 leading to regulator assembly 150 with inlet CGA fitting 148, regulator 150 and outlet CGA fitting 152. CGA fitting 154 will be coupled to the manifold. As above, it is helpful if the CGA fittings are selected to allow for only one assembly of the apparatus 310. The manifolds 160 are constructed to select one input gas to move to the gas blender valve 30. The only remaining difference is that with this many inputs for the valve 30 the face plate 162 will have effectively generic markings 164. The advantage of the embodiment of apparatus 310 is the added flexibility; however the noted disadvantage is the added complexity to the system and the loss of precise control markings for specific gas mixtures. The researcher must make some basic calculations to determine the Oxygen content (or Nitrogen content, or desired gas presence based upon the inputs) of the resulting mixture.

A further embodiment of the present invention is to provide an apparatus 510 as shown in FIG. 6 for providing an air and any subject gas of interest gas mixtures between 0% gas of interest and X% gas of interest to an animal. The apparatus 510 is similar to apparatus 110 described above wherein like reference numerals represent like elements, and these repeated elements need not be described again. The apparatus 510 includes a compressed air container or canister 72, replacing container 1 2 of apparatus 10. In this system the remaining canister 512 is a preformed mixture of air and the gas of interest at a concentration of X% gas of interest. The gas of interest may be any gas of interest (e.g. Carbon dioxide, carbon monoxide, methane, etc). The gas of interest and air canister can be made for the researcher by general gas suppliers as the gas supplier need only know the gas of interest and the maximum concentration X of this gas in order to supply the canister 512. The system will also include a face plate 532 with markings 534 from 0% to X% gas of interest.

In this embodiment the couplings for the canister 512 are “universal” in that it is expected the system is re-usable for different gases and different maximum concentrations. The container 512 includes a specific tank fitting 544 that couples to a regulator assembly 546 associated with the container 512. The regulator assembly 546 is equipped with a fitting 548 that matches “universal” fitting 544. The regulator assembly 546 includes a conventional gas regulator 550 for the nitrogen gas to regulate the pressure of the outgoing gas. The outlet of the regulator assembly 546 is a fitting 552 that attaches to a coupling hose with a fitting 554. The coupling hose has an opposite fitting 556 coupled to a fitting 558 found on a two-gas mechanical blending valve 30. The fittings associated with the canister 512 are not specific to a given gas, but should be different from air fittings to prevent the system from being assembled incorrectly. The face plate 532 should be removable or renewable (a stick on face plate 532 supplied by the gas supplier of the canister 512).

The system formed by apparatus 510 is intended to allow a researcher to quickly and easily perform research on gas blends of any relevant gas of interest. As a representative list, the following is a listing of hazardous gases that are detected by solid state sensors (with the levels of detection). This is also not intended to be an exhaustive listing, merely representative. This listing is repeated here as each of the identified “hazardous gases” can be the subject of a number of animal studies. The following listing should also demonstrate that the face plate markings may be in PPM (Parts per Million) of the subject gas, which can be preferred where the ranges of interest are relatively small.

| | |
|---|---|
| Acetic Acid | 100, 200 ppm |
| Acetone | 100, 200, 500, 1000, 5000 ppm; % LEL |
| Acetonitrile | 100 ppm |
| Acetylene | 50 ppm; % LEL; 3% by Volume |
| Acrolein | 50 ppm |
| (Acrylaldehyde) | |
| Acrylic Acid | 100 ppm |
| Acrylonitrile | 50, 60, 80, 100, 200, 500 ppm; % LEL |
| Allyl Alcohol | % LEL |
| Allyl Chloride | 200 ppm |
| Ammonia | 50, 70, 75, 100, 150, 200, 300, 400, |
| | 500, 1000, 2000, 2500, 4000, 5000 ppm; |
| | 1%, 2%, 10% by Vol., 10%, 25%, |
| | 100% LEL |
| Anisole | 100 ppm |
| Arsenic Pentafluoride | 5 ppm |
| Arsine | 1, 10 ppm |
| Benzene | 50, 75, 100, 1000 ppm; % LEL |
| Biphenyl | 50%, 100% LEL |
| Boron Trichloride | 500 ppm |
| Boron Trifluoride | 500 ppm |
| Bromine | 20 ppm |
| Butadiene | 50, 100, 3000 ppm; % LEL |
| Butane | 400, 1000 ppm; 100%, 200% LEL |
| Butanol | 1000 ppm, 100% LEL |
| Butene | 100% LEL |
| Butyl Acetate | 100 ppm; % LEL |
| Carbon Disulfide | 50, 60, 100 ppm; 5% by Volume |
| Carbon Monoxide | 50, 100, 150, 200, 250, 300, 500, 1000, |
| | 3000, 5000 ppm; 3%, 5% by Volume, % |
| | LEL |
| Carbon Tetrachloride | 50, 100, 10000 ppm |
| Cellosolve Acetate | 100 ppm |
| Chlorine | 10, 20, 50, 100, 200 ppm |
| Chlorine Dioxide | 10, 20 ppm |
| Chlorobutadiene | 100% LEL |
| Chloroethanol | 200 ppm |
| Chloroform | 50, 100, 200 ppm |
| Chlorotrifluoroethylene | 100% LEL |
| Cumene | 100% LEL |
| Cyanogen Chloride | 20 ppm |
| Cyclohexane | 100 ppm, 100% LEL |
| Cyclopentane | 50 ppm |
| Deuterium | 50%, 100% LEL |
| Diborane | 10, 50 ppm |
| Dibromoethane | 50 ppm |
| Dibutylamine | 100% LEL |
| Dichlorobutene | 1% by Volume |
| Dichloroethane (EDC) | 50, 100 ppm, % LEL |
| Dichlorofluoroethane | 100, 1000 ppm |
| Dichloropentadiene | 50 ppm |
| Dichlorosilane | 50, 100 ppm |
| Diesel Fuel | 50 ppm; 100% LEL |
| Diethyl Benzene | 100% LEL |
| Diethyl Sulfide | 10 ppm |
| Difluorochloroethane | 100% LEL |
| Difluoroethane (152A) | 100% LEL |
| Dimethyl Ether | 100% LEL |
| Dimethylamine (DMA) | 30, 50 ppm |
| Epichlorohydrin | 50, 100, 500, 1000 ppm |
| Ethane | 1000 ppm |
| Ethanol | 200, 1000, 2000 ppm; % LEL |
| Ethyl Acetate | 200, 1000 ppm; % LEL |
| Ethyl Benzene | 200 ppm; % LEL |
| Ethyl Chloride | 100 ppm; % LEL |
| Ethyl Chlorocarbonate | 1% by Volume |
| Ethyl Ether | 100, 800, 1000 ppm; % LEL |
| Ethylene | 100, 1000, 1200 ppm; % LEL |
| Ethylene Oxide | 5, 10, 20, 30, 50, 75, 100, 150, 200, |
| | 300, 1000, 1500, 2000, 3000 ppm; % |
| | LEL |
| Fluorine | 20, 100 ppm |
| Formaldehyde | 15, 50, 100, 500, 1000 ppm |
| Freon-11 | 1000, 2000, 5000 ppm |
| Freon-12 | 1000, 2000, 3000 ppm |
| Freon-22 | 100, 200, 500, 1000, 2000 ppm |
| Freon-113 | 100, 200, 500, 1000, 2000 ppm; 1% by |
| | Vol. |
| Freon-114 | 1000, 2000, 20000 ppm |
| Freon-123 | 1000 ppm |
| Fuel Oil or Kerosene | 100% LEL |
| Gasoline | 100, 1000, 2000, 20000 ppm; % LEL |
| Germane | 10, 50 ppm |
| Heptane | 1000 ppm, % LEL |
| Hexane | 50, 100, 200, 2000, 2500, 3000 ppm, % |
| | LEL |
| Hexene | % LEL |
| Hydrazine | 5, 10, 20, 100, 1000 ppm, 1% by |
| | Volume |
| Hydrogen | 50, 100, 200, 500, 1000, 2000, 5000 ppm; |
| | 3%, 5% by Vol., 2% to 100% LEL |
| Hydrogen Bromide | 50 ppm |
| Hydrogen Chloride | 50, 100, 200, 400, 500, 1000 ppm |
| Hydrogen Cyanide | 20, 30, 50, 100, 200, 1000, 10000 ppm |
| Hydrogen Fluoride | 20, 50, 100, 200 ppm |
| Hydrogen Sulfide | 5, 10, 20, 30, 50, 100, 300, 1000 ppm; |
| | % LEL |
| Isobutane | 1000, 3000 ppm, % LEL |
| Isobutylene | % LEL |
| Isopentane | 1000 ppm |
| Isoprene | % LEL |
| Isopropanol | 200, 400, 500, 1000 ppm; % LEL |
| JP4 | 1000 ppm; % LEL |
| JP5 | 1000, 5000 ppm, % LEL |
| Methane | 100, 200, 1000, 1500, 2000, 5000 ppm; |
| | 1%, 2% by Volume, 100%, 200% LEL |
| Methanol | 200, 300, 400, 500, 1000, 2000, 5000 ppm; |
| | 15%, 30%, 100% LEL |
| Methyl Acetate | 30 ppm |
| Methyl Acrylate | 60 ppm |
| Methyl Bromide | 20, 50, 60, 100, 500, 1000, 10000; |
| | 40,000 ppm |
| Methyl Butanol | % LEL |
| Methyl Cellosolve | % LEL |
| Methyl Chloride | 100, 200, 300, 2000, 10000 ppm; % LEL |
| Methyl Ethyl Ketone | 100, 500, 1000, 4000 ppm; 100% LEL |
| Methyl Hydrazine | 5 ppm |
| Methyl Isobutyl | 200, 500, 2000 ppm; 50%, 100% LEL |
| Ketone | |
| Methyl Mercaptan | 30 ppm |
| Methyl Methacrylate | 100 ppm; % LEL |
| Methyl-Tert Butyl | 100% LEL |
| Ether | |
| Methylene Chloride | 20, 100, 200, 300, 400, 500, 600, 1000, |
| | 2000, 3000, 5000 ppm; % LEL |
| Mineral Spirits | 200, 3000 ppm; % LEL |
| Monochlorobenzene | 100% LEL |
| Monoethylamine | 30, 100, 1000 ppm |
| Morpholine | 500 ppm |
| Naptha | 1000 ppm, 100% LEL |
| Natural Gas | 1000, 2000 ppm; 2%, 4% by Volume, % |
| | LEL |
| Nitric Oxide | 20, 50 ppm |
| Nitrogen Dioxide | 20, 50, 100 ppm |
| Nitrogen Trifluoride | 50, 500, 1000 ppm |
| Nonane | 2000 ppm |
| Pentane | 200, 1000 ppm, % LEL |
| Perchloroethylene | 200, 1000, 2000, 20000 ppm |
| Phenol | 100 ppm |
| Phosgene | 50 ppm |
| Phosphine | 3, 5, 10, 20, 30, 50 ppm |
| Phosphorus | 200 ppm |
| Oxychloride | |
| Picoline | % LEL |
| Propane | 100, 1000 ppm; 100% LEL |
| Propylene | 100, 200, 1000, 5000 ppm; % LEL |
| Propylene Oxide | 100 ppm,; % LEL |
| Silane | 10, 20, 50 ppm |
| Silicon Tetrachloride | 1000 ppm |
| Silicon Tetrafluoride | 1000 ppm |
| Styrene | 200, 300 ppm; % LEL |
| Sulfur Dioxide | 50, 100 ppm |
| Tetrahydrofuran | 200, 300, 1000 ppm; % LEL |
| Tetraline | 100 ppm |
| Toluene | 50, 100, 200, 500, 2000, 5000 ppm; % |
| | LEL |
| Toluene Diisocyanate | 15 ppm |
| Trichloroethane | 50, 100, 500, 1000 ppm; 1% by Volume |
| Trichloroethylene | 50, 100, 200, 300, 500, 1000, 2000 ppm; |
| | % LEL |
| Triethylamine (TEA) | 100 ppm |
| Trifluoroethanol | 25, 100 ppm |
| Trimethylamine (TMA) | 50 ppm |
| Tungsten Hexafluoride | 50 ppm |
| Turpentine | % LEL |
| Vinyl Acetate | 1000 ppm; % LEL |
| Vinyl Chloride | 20, 50, 100, 200, 400, 500, 1000, 4000, |
| | 10000 ppm; 10%, 100% LEL |
| Vinylidene Chloride | 50 ppm |
| Xylene | 100, 200, 300, 1000 ppm, 1% by |
| | Volume |

Although the present invention has been described with particularity herein, the scope of the present invention is not limited to the specific embodiment disclosed. It will be apparent to those of ordinary skill in the art that various modifications may be made to the present invention without departing from the spirit and scope thereof.

What is claimed is:

1. A method of supplying oxygen-nitrogen gas mixtures to an animal comprising the steps of:
(i) Providing a source of pressurized oxygen gas;
(ii) Providing a source of pressurized nitrogen gas;
(iii) Providing a mechanical two gas blending valve having a first gas inlet coupling, a second distinct gas inlet coupling and an outlet coupling configured for dispensing an oxygen-nitrogen mixture, the gas blending valve having a manual selector that can incrementally adjust the oxygen-nitrogen gas mixture within a range of mixtures, and a face plate on the blending valve including markings adjacent to the manual selector that are indicative of specific oxygen-nitrogen mixtures;
(iv) Providing a gas dispenser coupled to the outlet of the mechanical two gas blending valve;
(v) Providing an animal interface coupled to the gas dispenser and configured to deliver the gas mixture to the subject animal;
(vi) Coupling the source of oxygen gas to a gas inlet of the blending valve;
(vii) Coupling the source of nitrogen to a gas inlet of the blending valve;
(viii) Manually adjusting the selector to select a given oxygen-nitrogen gas mixture;
(ix) Simultaneously supplying oxygen and nitrogen containing gas to the blending valve, whereby the blending valve will dispense the selected oxygen-nitrogen gas mixture to the dispenser that will dispense the oxygen-nitrogen gas mixture to the animal through the animal interface.

2. A method of supplying oxygen-nitrogen gas mixtures to an animal according to claim 1 wherein the first gas inlet coupling is configured to be coupled only to the source of oxygen and the second gas inlet coupling is configured to be coupled only to the source of nitrogen.

3. A method of supplying oxygen-nitrogen gas mixtures to an animal according to claim 2 wherein the source of oxygen is a compressed oxygen container and the source of nitrogen is a compressed nitrogen container and wherein the face plate includes markings adjacent to the manual selector that are indicative of specific oxygen-nitrogen mixtures from 0% oxygen to 100% oxygen.

4. A method of supplying oxygen-nitrogen gas mixtures to an animal according to claim 2 wherein the source of oxygen is a compressed air container and the source of nitrogen is a compressed nitrogen container and wherein the face plate includes markings adjacent to the manual selector that are indicative of specific oxygen-nitrogen mixtures from 0% oxygen to 21% oxygen.

5. A method of supplying oxygen-nitrogen gas mixtures to an animal according to claim 2 further including three containers including a compressed air container, a compressed nitrogen container and a compressed oxygen container, wherein two of the three containers are selectively coupled to the blending valve to provide the source of oxygen and the source of nitrogen to the blending valve, whereby an oxygen-nitrogen selection provides for a gas mixture range of 0% Oxygen to 100% Oxygen on the manual selector, an air-nitrogen selection provides for a gas mixture range of 21% oxygen to 0% Oxygen, and an Air-Oxygen selection provides for a gas mixture range of 21% Oxygen to 100% Oxygen.

6. A method of supplying oxygen-nitrogen gas mixtures to an animal according to claim 1 wherein the source of oxygen is a compressed oxygen container and the source of nitrogen is a compressed nitrogen container and wherein the face plate includes markings adjacent to the manual selector that are indicative of specific oxygen-nitrogen mixtures from 0% oxygen to 100% oxygen.

7. A method of supplying oxygen-nitrogen gas mixtures to an animal according to claim 1 wherein the source of oxygen is a compressed air container and the source of nitrogen is a compressed nitrogen container and wherein the face plate includes markings adjacent to the manual selector that are indicative of specific oxygen-nitrogen mixtures from 0% oxygen to 21% oxygen.

8. A method of supplying oxygen-nitrogen gas mixtures to an animal according to claim 1 further including three containers including a compressed air container, a compressed nitrogen container and a compressed oxygen container, wherein two of the three containers are selectively coupled to the blending valve to provide the source of oxygen and the source of nitrogen to the blending valve, whereby an oxygen-nitrogen selection provides for a gas mixture range of 0% Oxygen to 100% Oxygen on the manual selector, an air-nitrogen selection provides for a gas mixture range of 21% oxygen to 0% Oxygen, and an Air-Oxygen selection provides for a gas mixture range of 21% Oxygen to 100% Oxygen.

9. An apparatus for supplying oxygen-nitrogen gas mixtures to an animal comprising:
(i) A source of pressurized oxygen gas;
(ii) A source of pressurized nitrogen gas;
(iii) A mechanical two-gas blending valve having a first gas inlet coupling, a second distinct gas inlet coupling and an outlet coupling configured for dispensing an oxygen-nitrogen mixture, the gas blending valve having a manual selector that can incrementally adjust the oxygen-nitrogen gas mixture within a range of mixtures, and a face plate on the blending valve including markings adjacent to the manual selector that are indicative of specific oxygen-nitrogen mixtures, wherein the gas inlets are coupled to the sources of oxygen and nitrogen, respectively;
(iv) A gas dispenser coupled to the outlet of the mechanical two-gas blending valve; and
(v) An animal interface coupled to the gas dispenser and configured to deliver the gas mixture to the subject animal.

10. An apparatus for supplying oxygen-nitrogen gas mixtures to an animal according to claim 9 wherein the animal interface is an animal housing configured to contain an animal therein, wherein the gas dispenser is coupled to the animal housing and configured to dispense the oxygen-nitrogen gas mixture to the animal housing.

11. An apparatus for supplying oxygen-nitrogen gas mixtures to an animal according to claim 10 wherein the source of oxygen is a compressed oxygen container and the source of nitrogen is a compressed nitrogen container and wherein the face plate includes markings adjacent to the manual selector that are indicative of specific oxygen-nitrogen mixtures from 0% oxygen to 100% oxygen.

12. An apparatus for supplying oxygen-nitrogen gas mixtures to an animal according to claim 10 wherein the source of oxygen is a compressed air container and the source of nitrogen is a compressed nitrogen container and wherein the face plate includes markings adjacent to the manual selector that are indicative of specific air-nitrogen mixtures from 0% oxygen to 21% oxygen.

13. An apparatus for supplying oxygen-nitrogen gas mixtures to an animal according to claim 10 further including three containers including a compressed air container, a compressed nitrogen container and a compressed oxygen container, wherein two of the three containers are selectively coupled to the blending valve to provide the source of oxygen and the source of nitrogen to the blending valve, whereby an oxygen-nitrogen selection provides for a gas mixture range of 0% Oxygen to 100% Oxygen on the manual selector, an air-nitrogen selection provides for a gas mixture range of 21% oxygen to 0% Oxygen, and an Air-Oxygen selection provides for a gas mixture range of 21% Oxygen to 100% Oxygen.

14. An apparatus for supplying oxygen-nitrogen gas mixtures to an animal according to claim 9 wherein the source of oxygen is a compressed oxygen container and the source of nitrogen is a compressed nitrogen container and wherein the face plate includes markings adjacent to the manual selector that are indicative of specific oxygen-nitrogen mixtures from 0% oxygen to 100% oxygen.

15. An apparatus for supplying oxygen-nitrogen gas mixtures to an animal according to claim 9 wherein the source of oxygen is a compressed air container and the source of nitrogen is a compressed nitrogen container and wherein the face plate includes markings adjacent to the manual selector that are indicative of specific air-nitrogen mixtures from 0% oxygen to 21% oxygen.

16. An apparatus for supplying oxygen-nitrogen gas mixtures to an animal according to claim 9 further including three containers including a compressed air container, a compressed nitrogen container and a compressed oxygen container, wherein two of the three containers are selectively coupled to the blending valve to provide the source of oxygen and the source of nitrogen to the blending valve, whereby an oxygen-nitrogen selection provides for a gas mixture range of 0% Oxygen to 100% Oxygen on the manual selector, an air-nitrogen selection provides for a gas mixture range of 21% oxygen to 0% Oxygen, and an Air-Oxygen selection provides for a gas mixture range of 21% Oxygen to 100% Oxygen.

17. A method of supplying an air and subject gas of interest gas mixtures between 0% gas of interest and X% gas of interest to an animal, wherein X is less than 100%, said method comprising the steps of:
(i) Providing a source of pressurized air;
(ii) Providing a source of pressurized gas of interest, wherein the source of gas of interest is a preformed mixture of X% gas of interest and the remainder air;
(iii) Providing a mechanical two gas blending valve having a first gas inlet coupling, a second distinct gas inlet coupling and an outlet coupling configured for dispensing an air-gas of interest mixture, the gas blending valve having a manual selector that can incrementally adjust the gas mixture within a range of mixtures, and a face plate on the blending valve including markings adjacent to the manual selector that are indicative of specific air-gas of interest mixtures between 0% and X% gas of interest concentrations;
(iv) Providing a gas dispenser coupled to the outlet of the mechanical two gas blending valve;
(v) Providing an animal interface coupled to the gas dispenser and configured to deliver the gas mixture to the subject animal;
(vi) Coupling the source of air to a gas inlet of the blending valve;
(vii) Coupling the source of gas of interest to a gas inlet of the blending valve;
(viii) Manually adjusting the selector to select a given gas mixture;
(ix) Simultaneously supplying air and gas of interest containing gas to the blending valve, whereby the blending valve will dispense the selected gas mixture to the dispenser that will dispense the gas mixture to the animal through the animal interface.

18. An apparatus for supplying an air and subject gas of interest gas mixtures between 0% gas of interest and X% gas of interest to an animal according to claim 17 wherein the animal interface is an animal housing configured to contain an animal therein, wherein the gas dispenser is coupled to the animal housing and configured to dispense the gas mixture to the animal housing.
